(19) United States
(12) Patent Application Publication
(10) Pub. No.: US 2016/0074550 A1
(43) Pub. Date: Mar. 17, 2016

(54) ODOR ADSORBENT

(71) Applicant: EVONIK INDUSTRIES AG, Essen (DE)

(72) Inventors: Christoph Loick, Tonisvorst (DE); Laurent Wattebled, Dusseldorf (DE)

(73) Assignee: EVONIK INDUSTRIES AG

(21) Appl. No.: 14/848,483

(22) Filed: Sep. 9, 2015

Related U.S. Application Data
(60) Provisional application No. 62/050,278, filed on Sep. 15, 2014.

(30) Foreign Application Priority Data
Sep. 15, 2014 (EP) 14184712.9

(51) Int. Cl. A61L 15/18; A61L 15/24; A61L 15/46; A61F 13/84

(52) U.S. Cl. CPC A61L 15/18; A61F 13/8405; A61L 15/24; A61L 15/46; A61F 2013/8408; A61L 2300/404; A61L 2300/11

(57) ABSTRACT

Described is an odor adsorbent, in particular for use in absorptive hygiene articles, comprising: a) peroxomonosulfuric acid and/or salts thereof and also b) zeolite. Also described are water-absorbing polymeric particles wherein the particles are laden with a mixture comprising a) peroxomonosulfuric acid and/or salts thereof and also b) zeolite, and wherein the water-absorbing polymeric particles preferably comprise a crosslinked polymer based on partially neutralized acrylic acid, and more particularly are surface-postcrosslinked.

The present invention resides in the field of odor adsorption, in particular in connection with water-absorbing polymeric particles and absorptive hygiene articles. The present invention relates to an odor adsorbent, to water-absorbing polymeric particles, to processes for producing odor adsorbents, to hygiene articles and to the use of an odor adsorbent for reducing, eliminating or preventing unpleasant odors.

Human excretions, such as urine, feces or else menses, can be collected with known hygiene articles, if necessary. Tampons or sanitary napkins can be used to collect menses, for example. Diapers can be worn to collect urine and/or feces, particularly for humans where excretion control has not yet been acquired, such as infants and toddlers, or is no longer adequate, as in the case of bowel and/or urinary incontinence.

These hygiene articles make possible the hygienic collection of the excretions and prevent, for example, soiling of clothing, bedding or the environment. State-of-the-art hygiene articles, such as diapers in particular, are often advantageously endowed with water-absorbing polymeric particles capable of binding and retaining appreciable amounts of liquid even under a confining pressure.

One issue which may arise in connection with the abovementioned excretions, even when the excretions are collected in hygiene articles, is the odor nuisance emanating from these excretions.

Urine, for example, will typically start to smell of ammonia over time. Depending on other factors such as dietary intake, illness or medication, there may be still other adverse odors associated with urine. The eating of asparagus, for instance, is known to be capable of causing a strong odor in human urine. This strong odor is due to the metabolism of aspartic acid in the human body. Other foods or drinks capable of leading to an undesirable odor for the urine include, for example, alcohols, onions, taste enhancers, aroma and flavor chemicals, coffee, spices, etc. Spicy food in particular can cause the urine to smell, since compounds present therein can often not be fully broken down by the human kidneys and therefore pass into the urine. Especially patients subject to certain dietary regimes and generally patients on particular medications or older people with declining renal function can produce urine with odor nuisance ingredients. Patients suffering from urinary incontinence are increasingly prone to excrete ureases which react with the urea in the urine and thus release toxic ammonia. There is also a disorder known as fish odor syndrome. It is based on increased excretion of quaternary ammonium compounds.

If, therefore, urine is collected in a hygiene article, such as a diaper for example, it will not be before long the odor nuisance ingredients of the urine will evolve an odor which is unpleasant to both the wearer of the diaper and his or her surroundings.

According to Rompp Chemie Lexikon, the concentration of urine ingredients is subject to physiological variations in that some substances are even excreted in a diurnally varying concentration, so particulars regarding the composition of urine are always based on the so-called 24 hour urine. Ingredients in the 24 hour urine of a healthy adult include, for example, urea (at an average of about 20 g), uric acid (about 0.5 g), creatinine (about 1.2 g), ammonia (about 0.5 g), amino acids (about 2 g), proteins (about 60 mg), reducing substances (about 0.5 g, of which about 70 mg of D-glucose or urea sugar), citric acid (about 0.5 g) and other organic acids and also some vitamins (C, B12 and others). Inorganic ions present include: Na⁺ (about 5.9 g), K⁺ (about 2.7 g), NH₄⁺ (about 0.8 g), Ca²⁺ (about 0.5 g), Mg²⁺ (about 0.4 g), Cl⁻ (about 8.9 g), PO₄³⁻ (about 4.1 g), SO₄²⁻ (about 2.4 g). Dry matter content is approximately between 50 and 72 g. Volatile components which have been identified for urine include alkylfurans, ketones, lactones, pyrroles, allyl isothiocyanate, dimethyl sulfone and others. The volatile components are usually molecules having a molar mass below about 1000 g/mol, which have a high vapor pressure.

Against this background, there is a fundamental need to reduce this odor nuisance, due to urine excretions in particular, as far as possible. There has therefore been no shortage of attempts to provide hygiene articles to address the odor issues referred to.

The problem addressed by the present invention in relation to this general background was specifically that of making possible the provision of hygiene articles that reduce malodor emanating from urine.

It was found, then, in the context of the present invention that the combined use of zeolite and peroxomonosulfuric acid and/or salts thereof makes the solution to the aforementioned problem possible.

One aspect of the present invention is accordingly an odor adsorbent, in particular for use in preferably absorptive hygiene articles, comprising:
a) peroxomonosulfuric acid and/or salts thereof,
b) zeolite.

This odor adsorbent has the advantage of being able to effectively reduce malodor emanating from urine, in particular as regards malodors due to foul-smelling breakdown products of urine, e.g. ammonia, but also due to those ingredients of urine which are immediately foul smelling on excretion, for example as a consequence of diet, illness or medication.

The use of the odor adsorbent of the present invention, in particular in preferably absorptive hygiene articles, thus makes possible the simple provision of hygiene articles that reduce malodor emanating from urine and hence minimize a potential odor nuisance.

It was found that, advantageously, components a) and b) of the odor adsorbent of the present invention cooperate synergistically, i.e. the effect of the whole is greater than the effect of the individual components a) plus b).

Peroxomonosulfuric acid and/or salts thereof are known per se. This acid is also called Caro's acid and corresponds to HO—SO₂—O—OH. The salts of peroxomonosulfuric acid are also known as caroates, examples being potassium hydrogenperoxomonosulfate (KHSO₅) and also sodium hydrogenperoxomonosulfate (NaHSO₅). The so-called potassium monopersulfate triple salt of the formula 2KHSO₅.KHSO₄.K₂SO₄ is particularly preferable in the present invention having regard to the present invention as a whole.

Zeolites are likewise known per se. Zeolites are crystalline aluminosilicates, numerous forms of which occur in nature but can also be produced synthetically. Examples of synthetic zeolites known from the general literature and also the patent literature are zeolite A, zeolite X, zeolite Y, zeolite P, zeolite L, mordenite, ZSM 5 and also ZSM 11.

Zeolites are compounds consisting of silicon oxide, aluminum oxide and a number of metal ions. Their composition reads M_{2/z}O.AI₂O₃.xSiO₂.yH₂O, where M is a mono or polyvalent metal, H and/or ammonium etc., z is the valency, x is from 1.8 to about 12 and y is from 0 to about 8. Structurally, zeolites consist of SiO and AIO₄ tetrahedra, linked via oxygen bridges. A channel system is formed, made up of equally constructed and equally sized interconnected voids. It is according to their pore opening that the zeolites are named, e.g. zeolite A (4.2 A), zeolite X (7.4 A). On heating, most zeolites release their water monotonously and without changing their crystalline structure. As a result, they can accommodate other compounds and then act as catalysts or ion exchangers for example. Zeolites further also exhibit a sifting effect by taking up molecules into the channel system of the lattice that have a smaller cross section than the pore openings. Larger molecules are excluded. Cations are needed to balance the negative charge of the AlO₄ tetrahedra in the aluminosilicate scaffold.

The synthesis of zeolites has been described in great detail, inter alia in: Zeolite Synthesis, ACS Symposium Series 398, Eds. M. L. Ocelli and H. E. Robson (1989) pages 2-7. The synthesis of hydrophobic zeolites as may be used with advantage in the context of this invention, for example, having a >100 silicon dioxide/aluminum oxide ratio for the scaffold and a high level of hydrothermal stability and resistance to aqueous alkaline solutions is disclosed in the patent application DE 19532500A1.

The designation “zeolite” in the context of the present invention comprehends in particular any microporous crystalline structure having pore diameters large enough to absorb at least one odorous organic molecular species, or compositions comprising such structures. The designation “zeolite” in the context of the present invention also comprehends in particular the so-called molecular sieves, which is the generally recognized designation for natural and synthetic zeolites possessing high adsorption capacity or cation-exchanging properties.

In one preferred embodiment of the invention, high-silicon zeolite is advantageously included in the odor adsorbent. High silicon in the context of the present invention is to be understood as meaning in particular that the silicon dioxide/aluminum oxide ratio is >10, preferably >20, more preferably >50 and yet more preferably >100. A silicon dioxide/aluminum oxide ratio >500 is most preferable.

Zeolites particularly suitable for the purposes of the present invention are available for example from UOP under the designation Abscents®. Suitable zeolites are for example also described in the patents U.S. Pat. No. 4,795,482, U.S. Pat. No. 5,013,335 and U.S. Pat. No. 4,855,154, which are hereby incorporated herein by reference.

Zeolites useful with particular advantage in this invention have a particle size of 0.1 to 50 μm, in particular of 0.5 to 10 μm. This corresponds to a preferred embodiment of the invention. This size range provides particularly good and effective adsorption of odor for the odorant molecules of bodily fluids in particular.

One suitable procedure for determining the particle size of a zeolite is a standard sieve analysis, although other techniques such as optical microscopy, image analysis, optical or resistivity zone sensing, or the like may also be appropriate depending on the size of the particles. The procedure for measuring particle size takes into consideration individual zeolite particles or agglomerates of such particles.

“Abscents® 3000” zeolite having a particle diameter of about 3 μm can be used in particular. “Abscents® 3000” zeolite is a zeolite from UOP Laboratories, Des Plaines, Ill., USA.

In a further preferred embodiment of the invention, the adsorbent of the present invention comprises from 0.0001 to 15 parts by weight, preferably from 0.005 to 3 parts by weight and especially from 0.01 to 0.5 part by weight of peroxomonosulfuric acid and/or salts thereof, and from 0.0005 to 5 parts by weight, preferably from 0.001 to 1.5 parts by weight and especially from 0.01 to 0.75 part by weight of zeolite, based on the overall amount of zeolite plus peroxomonosulfuric acid and/or salts thereof.

When the adsorbent of the present invention comprises water-absorbing polymeric particles in one preferred embodiment, it preferably comprises from 0.0001 to 15% by weight, preferably from 0.005 to 3% by weight and especially from 0.01 to 0.5% by weight of peroxomonosulfuric acid and/or salts thereof, especially in the form of the potassium monopersulfate triple salt, from 0.0005 to 5% by weight (preferably from 0.001 to 1.5% by weight, especially from 0.01 to 0.75% by weight) of zeolite, based on the overall amount formed from peroxomonosulfuric acid and/or salts thereof, from zeolite and from water-absorbing polymeric particles, while in such a case the adsorbent of the present invention advantageously includes at least 80% by weight, preferably at least 95% by weight and especially at least 99% by weight of water-absorbing polymeric particles.

The recited odor adsorbent of the present invention may comprise further substances, for example solvents, such as water in particular, additional adsorbents, e.g., activated carbon, cyclodextrins, etc. More particularly, the odor adsorbent of the present invention may be in solid form or in liquid form and also imported into the hygiene article in solid form or in liquid form.

It has proved to be very particularly advantageous for the requirements of the present invention for the odor adsorbent to be mingled with water-absorbing particles and/or fibers, in particular to be immobilized on water-absorbing particles and/or fibers.

When the odor adsorbent of the present invention is in an immobilized state on water-absorbing polymeric particles and, more particularly, is in solid pulverulent form, wherein the water-absorbing polymeric particles preferably comprise a crosslinked polymer based on partially neutralized acrylic acid, and, more particularly, are surface-postcrosslinked, this amounts to a preferred embodiment of the present invention. Immobilizing here is to be understood as meaning “spatially fixing”. More particularly, the immobilization corresponds to an at least partial coating of the water-absorbing polymeric particles with the odor adsorbent.

The aforementioned preferred embodiment, which resorts to water-absorbing polymeric particles, is notable for a particularly good odor-inhibiting effect and the odor adsorbents thus provided have a particularly high level of storage stability. The odor-inhibiting effect extends in particular also to malodorants having kakosmophoric groups in the form of amine derivatives and sulfur derivatives. It may also be considered a particular advantage of this embodiment for the odor-inhibiting effect to last for days following insultation with urine. This is important as regards, for example, urine-wet diapers subsequently disposed of in the garbage. The wet diapers and thus the garbage container can typically evolve a foul-smelling odor which gets stronger over days. The present invention and especially the aforementioned preferred embodiment, which resorts to water-absorbing polymeric particles, here even offer odor nuisance control lasting for days. As a result, the present invention offers comprehensive odor nuisance control in that it also extends to the post-use hygiene product disposed of in the garbage container.

Water-absorbing polymeric particles useful in the manufacture of diapers, tampons, sanitary napkins and other hygiene articles in particular are known per se and are also referred to as superabsorbents. The manufacture of water-absorbing polymeric particles is also known per se and is described at length for example in the monograph “Modern Superabsorbent Polymer Technology”, F. L. Buchholz and A T. Graham, Wiley-VCH, 1998, pages 71 to 103. The properties of water-absorbing polymeric particles are adjustable via the amount of crosslinker used, for example. As the amount of crosslinker increases, centrifuge retention capacity (CRC) decreases and absorption under pressure passes through a maximum. To improve their performance properties, for example saline flow conductivity (SFC) in the diaper and absorption under pressure, water-absorbing polymeric particles are generally subjected to surface postcrosslinking This increases the crosslink density of the particle surface, at least partly decoupling absorption under pressure and centrifuge retention capacity (CRC). This operation of surface postcrosslinking may be carried out in an aqueous gel phase. Preferably, however, dried, ground and sieved-off particles of (base) polymer are coated at the surface with a surface postcrosslinker, thermally surface-postcrosslinked and dried. Suitable crosslinkers for this include, for example, compounds capable of forming covalent bonds with at least two carboxylate groups of the water-absorbing polymeric particles.

Any known water-absorbing polymeric particles may be resorted to in the context and for the purposes of the present invention.

In one preferred embodiment of the invention, the odor adsorbent of the present invention advantageously includes at least 80% by weight, preferably 95% by weight and especially 99% by weight of water-absorbing polymeric particles, based on the overall mass of zeolite, peroxomonosulfuric acid and/or salts thereof and also water-absorbing polymeric particles.

Although any known water-absorbing polymeric particles may be resorted to in the context of this invention and their manufacture is well known, the manufacture of preferably useful water-absorbing polymeric particles will now be more particularly described because the herein below described water-absorbing polymeric particles are particularly preferable for use in the context of this invention and lead to particularly good results as regards attaining the desired object.

Water-absorbing polymeric particles useful in the context of the invention are obtainable for example by polymerizing a monomer solution or suspension comprising a) at least one ethylenically unsaturated acid-functional monomer which is optionally at least partly present in the form of a salt,
b) at least one crosslinker,
c) at least one initiator,
d) optionally one or more ethylenically unsaturated monomers copolymerizable with the monomers recited under a),
e) optionally one or more water-soluble polymers,
f) water and
g) optionally further additives and/or active substances. They are typically water-insoluble.

Useful monomers a) are preferably water-soluble, i.e. the solubility in water at 23° C. is typically at least 1 g/100 g of water, preferably at least 5 g/100 g of water, more preferably at least 25 g/100 g of water and most preferably at least 35 g/100 g of water.

Useful monomers a) are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

Further suitable monomers a) are, for example, ethylenically unsaturated sulfonic acids, such as styrenesulfonic acid and 2-acrylamido-2-methylpropanesulfonic acid (AMPS).

Impurities can have a considerable influence on the polymerization. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, an acrylic acid purified according to WO 2004/035514 A1 and comprising 99.8460% by weight of acrylic acid, 0.0950% by weight of acetic acid, 0.0332% by weight of water, 0.0203% by weight of propionic acid, 0.0001% by weight of furfural, 0.0001% by weight of maleic anhydride, 0.0003% by weight of diacrylic acid and 0.0050% by weight of hydroquinone monomethyl ether. The proportion of acrylic acid and/or salts thereof in the total amount of monomers a) is preferably at least 50 mol %, more preferably at least 90 mol %, most preferably at least 95 mol %. The monomers a) may typically comprise polymerization inhibitors, preferably hydroquinone monoethers, as storage stabilizers.

The monomer solution used may comprise preferably up to 250 ppm by weight, preferably at most 130 ppm by weight, more preferably at most 70 ppm by weight, and preferably at least 10 ppm by weight, more preferably at least 30 ppm by weight and especially around 50 ppm by weight, of hydroquinone monoethers, each based on the unneutralized monomer a). For example, the monomer solution can be prepared by using an ethylenically unsaturated monomer bearing acid groups with an appropriate content of hydroquinone monoether.

Preferred hydroquinone monoethers are hydroquinone monomethyl ether (MEHQ) and/or alpha-tocopherol (vitamin E).

Suitable crosslinkers b) are for example compounds having at least two groups suitable for crosslinking Such groups are, for example, ethylenically unsaturated groups which can be polymerized free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b) for example.

Crosslinkers b) are preferably compounds having at least two polymerizable groups which can be polymerized free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/032962 A2.

Preferred crosslinkers b) are pentaerythrityl triallyl ether, tetraalloxyethane, methylenebismethacrylamide, 15-tuply ethoxylated trimethylolpropane triacrylate, polyethylene glycol diacrylate, trimethylolpropane triacrylate and triallylamine. Very particularly preferred crosslinkers b) are the polyethoxylated and/or -propoxylated glycerols which have been esterified with acrylic acid or methacrylic acid to give di- or triacrylates, as described, for example, in WO 2003/104301 A1.

Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably 0.05% to 1.5% by weight, more preferably 0.1% to 1% by weight, most preferably 0.3% to 0.6% by weight, based in each case on monomer a). With rising crosslinker content, centrifuge retention capacity (CRC) falls and the absorption under pressure passes through a maximum.

Initiators c) used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany).

Ethylenically unsaturated monomers d) copolymerizable with the ethylenically unsaturated monomers a) bearing acid groups are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

The water-soluble polymers e) used may, for example, be polyvinyl alcohol, polyvinylpyrrolidone, starch, starch derivatives, modified cellulose, such as methyl cellulose or hydroxyethyl cellulose, gelatin, polyglycols or polyacrylic acids, preferably starch, starch derivatives and modified cellulose.

Typically, an aqueous monomer solution can be used. The water content of the monomer solution is preferably from 40% to 75% by weight, more preferably from 45% to 70% by weight and most preferably from 50% to 65% by weight. It is also possible to use monomer suspensions, i.e. monomer solutions with excess monomer a), for example sodium acrylate. As the water content rises, the energy expenditure in the subsequent drying rises and, as the water content falls, the heat of polymerization can only be removed inadequately.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on a belt is described, for example, in DE 38 25 366 A1 and U.S. Pat. No. 6,241,928. Polymerization in a belt reactor forms a polymer gel which has to be comminuted in a further process step, for example in an extruder or kneader. However, it is also possible to dropletize an aqueous monomer solution and to polymerize the droplets obtained in a heated carrier gas stream. It is possible here to combine the process steps of polymerization and drying, as described in WO 2008/040715 A2 and WO 2008/052971 A1.

The acid groups of the resulting polymer gels have typically been partly neutralized. Neutralization is preferably carried out at the monomer stage. This is typically accomplished by mixing in the neutralizing agent as an aqueous solution or preferably also as a solid. The degree of neutralization is preferably from 25 to 85 mol %, for “acidic” polymer gels more preferably from 30 to 60 mol %, most preferably from 35 to 55 mol %, for “neutral” polymer gels more preferably from 65 to 80 mol %, most preferably from 70 to 75 mol %, and the customary neutralizing agents may be used, preferably alkali metal hydroxides, alkali metal oxides, alkali metal carbonates or alkali metal bicarbonates and also mixtures thereof. Ammonium salts such as the salt of triethanolamine can also be used instead of alkali metal salts. Particularly preferred alkali metals are sodium and potassium, but very particular preference is given to sodium hydroxide, sodium carbonate or sodium hydrogencarbonate and also mixtures thereof.

However, it is also possible to carry out neutralization after the polymerization, at the stage of the polymer gel forming in the polymerization. It is also possible to neutralize up to 40 mol %, preferably 10 to 30 mol % and more preferably 15 to 25 mol % of the acid groups before the polymerization by adding a portion of the neutralizing agent directly to the monomer solution and setting the desired final degree of neutralization only after the polymerization, at the polymer gel stage. When the polymer gel is at least partly neutralized after the polymerization, the polymer gel is preferably comminuted mechanically, for example by means of an extruder, in which case the neutralizing agent can be sprayed, sprinkled or poured on and then carefully mixed in. For this purpose, the gel material obtained can be extruded several times more for homogenization.

The resulting polymer gel can preferably be dried with a belt dryer until the residual moisture content is preferably 0.5 to 15% by weight, more preferably 1 to 10% by weight and most preferably 2 to 8% by weight, the residual moisture content being determined by EDANA (European Disposables and Nonwovens Association) recommended test method No. WSP 230.2-05 “Moisture Content”. The EDANA test methods are obtainable, for example, from EDANA, Avenue Eugene Plasky 157, B-1030 Brussels, Belgium.

In the case of too high a residual moisture content, the dried polymer gel has too low a glass transition temperature T_{G} and can then be processed further only with difficulty. In the case of too low a residual moisture content, the dried polymer gel can be too brittle and, in the subsequent comminution steps, undesirably large amounts of polymer particles with too low a particle size (“fines”) can be obtained. The solids content of the gel before the drying is preferably from 25% to 90% by weight, more preferably from 35% to 70% by weight and most preferably from 40% to 60% by weight. However, a fluidized bed dryer or a paddle dryer may optionally also be used for drying purposes.

Thereafter, the dried polymer gel is preferably ground and classified, and the apparatus used for grinding may typically be single or multistage roll mills, preferably two- or three-stage roll mills, pin mills, hammer mills or vibratory mills.

The mean particle size of the polymer particles removed as the product fraction is preferably at least 200 μm, more preferably from 250 to 600 μm and very particularly from 300 to 500 μm. The mean particle size of the product fraction may be determined by means of EDANA (European Disposables and Nonwovens Association) recommended test method No. WSP 220.2-05 “Particle Size Distribution”, where the proportions by mass of the screen fractions are plotted in cumulated form and the mean particle size is determined graphically. The median particle size here is the mesh size value at which a cumulative 50% by weight is found.

The proportion of particles having a particle size of at least 150 μm is preferably at least 90% by weight, more preferably at least 95% by weight, most preferably at least 98% by weight.

Polymer particles having too low a particle size lower the saline flow conductivity (SFC). Therefore, the proportion of excessively small polymer particles (“fines”) should be small. Excessively small polymer particles are therefore typically separated off and recycled into the process. This is preferably done before, during or immediately after the polymerization, i.e. before the drying of the polymer gel. The excessively small polymer particles can be moistened with water and/or aqueous surfactant before or during the recycling.

It is also possible to remove excessively small polymer particles in later process steps, for example after the surface postcrosslinking or another coating step. In this case, the excessively small polymeric particles recycled are surface postcrosslinked or coated in another way, for example with fumed silica.

If a kneading reactor is used for polymerization, the excessively small polymer particles are preferably added during the last third of the polymerization.

If the excessively small polymer particles are added at a very early stage, for example directly to the monomer solution, this lowers the centrifuge retention capacity (CRC) of the resulting water-absorbing polymer particles. However, this can be compensated, for example, by adjusting the amount of crosslinker b) used.

If the excessively small polymer particles are added at a very late stage, for example not until an apparatus connected downstream of the polymerization reactor, for example an extruder, the excessively small polymer particles can be incorporated into the resulting polymer gel only with difficulty. Insufficiently incorporated, excessively small polymer particles are, however, detached again from the dried polymer gel during the grinding, are therefore removed again in the course of classification and increase the amount of excessively small polymer particles to be recycled.

The proportion of particles having a particle size of at most 850 μm is preferably at least 90% by weight, more preferably at least 95% by weight, most preferably at least 98% by weight.

The proportion of particles having a particle size of at most 600 μm is preferably at least 90% by weight, more preferably at least 95% by weight, most preferably at least 98% by weight.

Polymer particles having too high a particle size lower the free swell rate. Therefore, the proportion of excessively large polymer particles should likewise be small. Excessively large polymer particles are therefore typically separated off and recycled into the grinding of the dried polymer gel.

To further improve the properties, the polymer particles can be surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and

EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230. Additionally described as suitable surface postcrosslinkers are cyclic carbonates in DE 40 20 780 C1, 2-oxazolidone and derivatives thereof, such as 2-hydroxyethyl-2-oxazolidone, in DE 198 07 502 A1, bis- and poly-2-oxazolidones in DE 198 07 992 C1, 2-oxotetrahydro-1,3-oxazine and derivatives thereof in DE 198 54 573 A1, N-acyl-2-oxazolidones in DE 198 54 574 A1, cyclic ureas in DE 102 04 937 A1, bicyclic amido acetals in DE 103 34 584 A1, oxetanes and cyclic ureas in EP 1 199 327 A2 and morpholine-2,3-dione and derivatives thereof in WO 2003/031482 A1. Preferred surface postcrosslinkers are ethylene carbonate, ethylene glycol diglycidyl ether, reaction products of polyamides with epichlorohydrin and mixtures of propylene glycol and 1,4-butanediol. Very particularly preferred surface postcrosslinkers are 2-hydroxyethyloxazolidin-2-one, oxazolidin-2-one and 1,3-propanediol.

In addition, it is also possible to use surface postcrosslinkers which comprise additional polymerizable ethylenically unsaturated groups, as described in DE 37 13 601 A1.

The amount of surface postcrosslinker is preferably 0.001% to 2% by weight, more preferably 0.02% to 1% by weight and most preferably 0.05% to 0.75% by weight, based in each case on the polymer particles.

In a preferred embodiment of the present invention, polyvalent cations are applied to the particle surface in addition to the surface postcrosslinkers before, during or after the surface postcrosslinking The polyvalent cations usable are, for example, divalent cations such as the cations of zinc, magnesium, calcium and strontium, trivalent cations such as the cations of aluminum, tetravalent cations such as the cations of titanium and zirconium. Possible counterions are, for example, chloride, bromide, sulfate, hydrogensulfate, carbonate, hydrogencarbonate, nitrate, phosphate, hydrogenphosphate, dihydrogenphosphate and carboxylate, such as acetate and lactate. Aluminum sulfate and aluminum lactate are preferred. Apart from metal salts, it is also possible to use polyamines as polyvalent cations. The amount of polyvalent cation used is, for example, 0.001% to 1.5% by weight, preferably 0.005% to 1% by weight and more preferably 0.02% to 0.8% by weight, based in each case on the polymer particles.

The surface postcrosslinking is typically performed in such a way that a solution of the surface postcrosslinker is applied to, preferably sprayed onto, the dried polymer particles. After the spray application, the polymer particles coated with surface postcrosslinker are dried thermally, and the surface postcrosslinking reaction can take place either before or during the drying.

The spray application of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers have a vertically mounted mixing shaft. Suitable mixers are, for example, horizontal Pflugschar® plowshare mixers (Gebr. Lödige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; USA) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. It is possible to adjust the penetration depth of the surface postcrosslinker into the polymer particles via the content of nonaqueous solvent or total amount of solvent.

When exclusively water is used as the solvent, a surfactant is advantageously added. This improves the wetting characteristics and reduces the tendency to form lumps. However, preference is given to using solvent mixtures, for example isopropanol/water, 1,3-propanediol/water and propylene glycol/water, where the mixing ratio in terms of mass is preferably from 20:80 to 40:60.

The thermal drying is preferably carried out in contact dryers, more preferably paddle dryers, most preferably disk dryers. Suitable dryers are, for example, Hosokawa Bepex® Horizontal Paddle Dryer (Hosokawa Micron GmbH; Leingarten; Germany), Hosokawa Bepex® Disc Dryer (Hosokawa Micron GmbH; Leingarten; Germany) and Nara Paddle Dryer (NARA Machinery Europe; Frechen; Germany). Moreover, fluidized bed dryers may also be used. The drying can be effected in the mixer itself, by heating the jacket or blowing in warm air. Equally suitable is a downstream dryer, for example a shelf dryer, a rotary tube oven or a heatable screw. It is particularly advantageous to effect mixing and drying in a fluidized bed dryer.

Preferred drying temperatures are in the range of 100 to 250° C., preferably 120 to 220° C., more preferably 130 to 210° C. and most preferably 150 to 200° C. The preferred residence time at this temperature in the reaction mixer or dryer is preferably at least 10 minutes, more preferably at least 20 minutes, most preferably at least 30 minutes, and typically at most 60 minutes.

Subsequently, the surface postcrosslinked polymer particles can be classified again, with removal of excessively small and/or excessively large polymer particles and recycling into the process.

The surface-postcrosslinked polymeric particles may be aftertreated to further improve their properties, by coating and/or remoistening.

The optional remoisturizing is preferably performed at 30 to 80° C., more preferably at 35 to 70° C., most preferably at 40 to 60° C. At excessively low temperatures, the water-absorbing polymer particles tend to form lumps, and, at higher temperatures, water already evaporates to a noticeable degree. The amount of water used for remoisturizing is preferably from 1 to 10% by weight, more preferably from 2 to 8% by weight and most preferably from 3 to 5% by weight. The remoisturizing increases the mechanical stability of the polymer particles and reduces their tendency to static charging. Suitable coatings for improving the free swell rate and permeability (SFC) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the polymer particles are, for example, fumed silica, such as Aerosil® 200, and surfactants, such as Span® 20.

The water-absorbing polymer particles have a moisture content of preferably 1 to 15% by weight, more preferably 2 to 10% by weight and most preferably 3 to 5% by weight, the moisture content preferably being determined by EDANA (European Disposables and Nonwovens Association) recommended test method No. WSP 230.2-05 “Moisture Content”.

The water-absorbing polymer particles in the present invention have a centrifuge retention capacity (CRC) of typically at least 15 g/g, preferably at least 20 g/g, more preferably at least 22 g/g, especially preferably at least 24 g/g and most preferably at least 26 g/g. The centrifuge retention capacity (CRC) of the water-absorbing polymer particles is typically less than 60 g/g. The centrifuge retention capacity (CRC) is preferably determined by EDANA (European Disposables and Nonwovens Association) recommended test method No. WSP 241.2-05 “Centrifuge Retention Capacity”. The water-absorbing polymeric particles have an absorption under a pressure of 49.2 g/cm² of typically at least 15 g/g, preferably at least 20 g/g, more preferably at least 22 g/g, especially preferably at least 24 g/g and most preferably at least 26 g/g. The absorption under a pressure of 49.2 g/cm² of the water-absorbing polymer particles is typically less than 35 g/g. The absorption under a pressure of 49.2 g/cm² may be determined similarly to EDANA's recommended test method No. WSP 242.2-05 “Absorption Under Pressure, Gravimetric Determination”, setting a pressure of 49.2 g/cm².

A further entity provided by the present invention comprises water-absorbing polymeric particles wherein the particles are laden, in particular at least partially coated, with a mixture comprising
a) peroxomonosulfuric acid and/or salts thereof, in particular potassium monopersulfate triple salt
b) zeolite,

wherein the water-absorbing polymeric particles preferably comprise a crosslinked polymer based on partially neutralized acrylic acid and more particularly are surface-postcrosslinked.

This aspect enables outstanding malodor inhibition within the meaning of the invention and also exhibits a high level of stability in storage.

In one preferred embodiment of the invention, the amount of zeolite is from 0.0005 to 5% by weight, preferably from 0.001 to 1.5% by weight and especially from 0.01 to 0.75% by weight and the amount of peroxomonosulfuric acid and/or salts thereof is from 0.0001 to 15% by weight, preferably from 0.005 to 3% by weight and especially from 0.01 to 0.5% by weight, all based on the water-absorbing polymeric particles laden with zeolite and peroxomonosulfuric acid and/or salts thereof.

The present invention further provides a process for producing odor adsorbents of the present invention, as described above, or for producing water-absorbing polymeric particles, as described above, which comprises contacting, preferably coating, water-absorbing polymeric particles with a preferably aqueous solution of peroxomonosulfuric acid and/or salts thereof together with zeolite, wherein the contacting is effected by spraying in particular.

An aftertreating additive, in particular a dust-reducing agent, may advantageously also be admixed in the process of producing the water-absorbing polymeric particles of the present invention together with the contacting with peroxomonosulfuric acid and/or salts thereof and also zeolite.

Suitable dust-reducing agents include polyglycerols, polyethylene glycols, polypropylene glycols, random or block copolymers of ethylene oxide and propylene oxide. Further dustproofing agents suitable for this further include polyethoxylates or polypropoxylates of polyhydroxyl compounds, such as glycerol, sorbitol, trimethylolpropane, trimethylolethane and pentaerythritol. Examples thereof are n-tuply ethoxylated trimethylolpropane or glycerol, where n is an integer between 1 and 100. Further examples are block copolymers, such as altogether n-tuply ethoxylated and then m-tuply propoxylated trimethylolpropane or glycerol, where n is an integer between 1 and 40 and m is an integer between 1 and 40. The order of the blocks may also be the other way round. Dust-reducing agents can also be water diluted.

The present invention further provides a hygiene article, preferably an absorptive hygiene article, in particular a sanitary napkin, a tampon, an incontinence aid, preferably a diaper, comprising an odor adsorbent of the present invention, as described above, or water-absorbing polymeric particles of the present invention, as described above.

The hygiene articles typically contain a water-impervious backsheet, a water-pervious top sheet, and between them an absorbent core composed of the inventive water-absorbing polymer particles and fibers, preferably cellulose. The proportion of the inventive water-absorbing polymer particles in the absorbent core is preferably 20% to 100% by weight, more preferably 50% to 100% by weight.

The present invention further provides for the use of an odor adsorbent of the present invention or of water-absorbing polymeric particles of the present invention for reducing or eliminating unpleasant odors preferably emanating from human excretion products, preferably urine, in particular for reducing or eliminating such unpleasant odors resulting from foul-smelling components already present in the human excretion product, preferably urine, at the time of excretion.

The present invention further provides for the use of an odor adsorbent of the present invention or of water-absorbing polymeric particles of the present invention for preventing the formation of unpleasant odors resulting from breakdown products of human excretion products, preferably urine, in particular for inhibiting unpleasant odor due to ammonia.

Reducing the unpleasant-smelling odorants in the use of a hygiene article in a hygiene application, in particular in the gas space above the solid/liquid phase of a hygiene article in a hygiene application, is a preferred embodiment of the invention in the context of the aforementioned uses.

### EXAMPLES

Procedures

Odor control for organic molecules; SPME-GC

0.50 g of the superabsorbent to be determined was accurately weighed into a 200 ml conical flask and spiked with 11.0 g of odor cocktail. The flask was sealed with a threaded adapter having a septum and stored in a climate-controlled cabinet at 37° C. for 16 h to establish equilibrium in the vapor space. The SPME phase was then introduced into the vapor space for 30 min and then injected directly. The decrease or the reduction in concentration of the odorant was determined in relation to the corresponding reference sample in %. The decrease was computed from the averaged areas of the chromatograms. The standard deviation of this procedure is below 5%.

Instrument Parameters:

| | |
|---|---|
| Flask: | 200 ml conical flask with NS 29 = volume 255 ml |
| | with threaded adapter and septum |
| Phase: | Supelco Carboxen/PDMS-black |
| GC column: | RESTEK Corp. RTX-50, 30 m, 0.53 |
| GC: | Hewlett Packard 5890 |
| Heating rates: | 7 min. 30° C. |
| | 10° C./min. to 180° C. |
| | 30° C./min. to 250° C. |

Odor Cocktail:

Concentrations of odorants

| | | |
|---|---|---|
| Diacetyl | 250 | ppb |
| 3-Methylbutanal | 40 | ppb |
| Dimethyl trisulfide | 100 | ppb |
| p-Cresol | 20 | ppm |

The odor cocktail is designed for malodor simulation.

Method:

Polymer Material (Powder A)

A monomer solution consisting of 300 g of acrylic acid neutralized to an extent of 60 mol % with 200.2 g of 50% NaOH, 474.8 g of water, 1.62 g of polyethylene glycol-300 diacrylate, 0.89 g of monoallyl polyethylene glycol-450 monoacrylate was freed of the dissolved oxygen by degassing with nitrogen and cooled to the start temperature of about 4° C. After the start temperature had been attained, an initiator solution (0.3 g of sodium peroxodisulfate in 10 g of water, 0.07 g of 35% hydrogen peroxide solution in 10 g of water and 0.015 g of ascorbic acid in 2 g of water) was added. An exothermic polymerization reaction took place. The adiabatic end temperature was about 100° C. The hydrogel formed was comminuted using a laboratory mincer (5 mm breaker plate). The minced sample was subsequently dried in a laboratory circulating air drying cabinet at 170° C. for 90 minutes. The dried polymer was first coarsely crushed and then ground by means of an SM100 cutting mill having an aperture size of 2 mm, and sieved to a powder having a particle size of 150 to 850 μm. 100 g of the powder were coated with a solution of 1.0 g of ethylene carbonate and 3.0 g of deionized water. This was done by applying the solution with a syringe (0.45 mm cannula) to the polymer powder present in the mixer. The coated powder was then heated in a drying cabinet at 170° C. over a period of 90 minutes.

### Example 1

### Reference Sample

The reference sample used was powder A without further additional treatment.

### Example 2

100 g of powder A were admixed with 1.25 g of a 20% aqueous potassium peroxomonosulfate triple salt (Caro's acid) solution and with 0.75 g of Abscents® 3000 and homogenized on an bottle-inverting mixer for about 2 hours. “Abscents® 3000” zeolite is a zeolite from UOP Laboratories, Des Plaines, Ill., USA.

### Example 3

100 g of powder A were admixed with 1.25 g of a 20% aqueous potassium peroxomonosulfate triple salt (Caro's acid) solution and with 0.50 g of Abscents® 3000 and homogenized on an bottle-inverting mixer for about 2 hours.

### Example 4

100 g of powder A were admixed with 1.00 g of a 20% aqueous potassium peroxomonosulfate triple salt (Caro's acid) solution and with 1.00 g of Abscents® 3000 and homogenized on an bottle-inverting mixer for about 2 hours.

### Example 5

100 g of powder A were admixed with 0.75 g of a 20% aqueous potassium peroxomonosulfate triple salt (Caro's acid) solution and with 1.00 g of Abscents® 3000 and homogenized on an bottle-inverting mixer for about 2 hours.

### Example 6

100 g of powder A were admixed with 0.50 g of a 20% aqueous potassium peroxomonosulfate triple salt (Caro's acid) solution and with 1.00 g of Abscents® 3000 and homogenized on an bottle-inverting mixer for about 2 hours.

| Percentage | | | | | |
|---|---|---|---|---|---|
| decreases | | | | | |
| versus | | | | | |
| Example 1 | Example 2 | Example 3 | Example 4 | Example 5 | Example 6 |
| Diacetyl | 100.00% | 100.00% | 100.00% | 100.00% | 100.00% |
| 3-Methyl- | 93.63% | 81.62% | 96.09% | 96.02% | 96.10% |
| butanal | | | | | |
| DMTS | 96.48% | 96.86% | 97.18% | 96.37% | 98.48% |
| p-Cresol | 26.35% | 14.80% | 27.79% | 22.87% | 26.62% |

### Example 7

100 g of powder A were admixed with 0.25 g of a 20% aqueous potassium peroxomonosulfate triple salt (Caro's acid) solution and with 0.75 g of Abscents® 3000 and homogenized on an bottle-inverting mixer for about 2 hours.

### Example 8

100 g of powder A were admixed with 0.10 g of a 20% aqueous potassium peroxomonosulfate triple salt (Caro's acid) solution and with 1.00 g of Abscents® 3000 and homogenized on an bottle-inverting mixer for about 2 hours.

In addition to the previously described odorants, Examples 7 and 8 were tested with the following well-known malodors:

Concentrations of odorants

| | | |
|---|---|---|
| Pyrrol | 2 | ppm |
| Furfural mercaptan | 500 | ppb |
| Indol | 2.5 | ppm |
| Dimethyl disulphide | 100 | ppb |

| Percentage | | |
|---|---|---|
| decreases versus | | |
| Example 1 | Example 7 | Example 8 |
| Pyrrol | 82.42% | 42.15% |
| Furfural mercaptan | 99.31% | 96.75% |
| (S)-(+) Carvon | 32.54% | 55.79% |
| Indol | 81.38% | 52.67% |
| Dimethy disulfide | 97.98% | 98.48% |
| Diacetyl | 100.00% | 100.00% |
| 3-Methyl-butanal | 92.13% | 95.55% |
| DMTS | 94.10% | 95.29% |
| p-Cresol | 19.25% | 22.60% |

1. An odor adsorbent, in particular for use in absorptive hygiene articles, comprising:
a) peroxomonosulfuric acid and/or salts thereof,
b) zeolite.

2. Adsorbent according to claim 1, characterized in that it comprises from 0.0001 to 15 parts by weight of peroxomonosulfuric acid and/or salts thereof, and from 0.0005 to 5 parts by weight of zeolite, based on the overall amount of zeolite plus peroxomonosulfuric acid and/or salts thereof.

3. Adsorbent according to claim 1, wherein the zeolites have a particle size of 0.1 to 50 μm.

4. Adsorbent according to any of claim 1, wherein it is in an immobilized state on water-absorbing polymeric particles and more particularly is in solid pulverulent form, wherein the water-absorbing polymeric particles preferably comprise a crosslinked polymer based on partially neutralized acrylic acid, and more particularly are surface-postcrosslinked.

5. Adsorbent according to claim 1, wherein it includes at least 80% by weight of water-absorbing polymeric particles, based on the overall mass of zeolite, peroxomonosulfuric acid and/or salts thereof and also water-absorbing polymeric particles.

6. A water-absorbing polymeric particle, wherein the water-absorbing polymeric particle are laden, in particular at least partially coated, with a mixture comprising
a) peroxomonosulfuric acid and/or salts thereof
b) zeolite,
wherein the water-absorbing polymeric particle comprise a crosslinked polymer based on partially neutralized acrylic acid and more particularly are surface-postcrosslinked.

7. The water-absorbing polymeric particle according to claim 6, wherein the amount of zeolite is from 0.0005 to 5% by weight and also in that the amount of peroxomonosulfuric acid and/or salts thereof is from 0.0001 to 15% by weight, all based on water-absorbing polymeric particles laden with zeolite and peroxomonosulfuric acid and/or salts thereof.

8. A process for producing odor adsorbents wherein water-absorbing polymeric particles are contacted, with an aqueous solution of peroxomonosulfuric acid and/or salts thereof together with zeolite, wherein the contacting is effected by spraying in particular.

9. A hygiene article comprising an odor adsorbent according to claim 1.

10. Use of an odor adsorbent according to claim 1 for reducing or eliminating unpleasant odors preferably emanating from human excretion products, preferably urine, in particular for reducing or eliminating such unpleasant odors resulting from foul-smelling components already present in the human excretion product, preferably urine, at the time of excretion.

11. Use of an odor adsorbent according to claim 1 for preventing the formation of unpleasant odors resulting from breakdown products of human excretion products, preferably urine, in particular for inhibiting unpleasant odor due to ammonia.

12. Use according to claim 10 for reducing unpleasant-smelling odorants in the use of a hygiene article in a hygiene application, in particular in the gas space above the solid/liquid phase of a hygiene article in a hygiene application.
